(19) United States
(12) Patent Application Publication
(10) Pub. No.: US 2009/0208495 A1
(43) Pub. Date: Aug. 20, 2009

(54) ANTI-TUMOR EFFECTIVE PARAMYXOVIRUS

(75) Inventors: Rudolf Beier, Berlin (DE); Florian Puehler, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER SCHERING PHARMA AG, Berlin (DE)

(21) Appl. No.: 12/368,330

(22) Filed: Feb. 10, 2009

Related U.S. Application Data
(60) Provisional application No. 61/029,650, filed on Feb. 19, 2008.

(30) Foreign Application Priority Data
Feb. 14, 2008 (EP) 08075121.7

(51) Int. Cl. A61K 39/395; A61K 35/76

(52) U.S. Cl. 424/133.1; 424/93.6

(57) ABSTRACT

Paramyxovirus from the group APMV3, APMV4, APMV5, APMV6, APMV7, APMV8, APMV9, Mapueravirus and Fer-de-Lance virus are described, which can be used for the production of a medicament for the treatment of tumors. The virus has a selectivity to kill human tumor cells but not human normal differentiated and human normal proliferating cells at the same dose. By genetic engineering the virus can be modified in such a way that one or more genes are added or are replaced by the homologous genes of a related paramyxovirus. By that method the anti-tumor activity of the resulting chimeric virus is enhanced compared to the parental virus.

This application claims the benefit of the filing date of U.S. Provisional Application Ser. No. 61/029,650 filed Feb. 19, 2008, which is incorporated by reference herein.

The present invention relates to the treatment of tumors by administration of live animal paramyxovirus. The virus has a selectivity to kill human tumor cells but not human normal differentiated and human normal proliferating cells at the same dose. By genetic engineering the virus can be modified in such a way that one or more genes are replaced by the homologous genes of a related paramyxovirus. Alternatively these homologous genes are inserted as additional transgenes into the virus genome. By that method the anti-tumor activity of the resulting chimeric virus is enhanced compared to the parental virus.

The method of genetic engineering also allows the modification of the F protein cleavage site to be recognized by an enzyme that is expressed in tumor cells.

From the state of the art it is known that oncolytic viruses can be used as cancer therapeutics. Oncolytic viruses in general for the treatment of tumors are reviewed in (Chiocca, 2002). An actual list of oncolytic viruses is published in (Vaha-Koskela, Heikkila, and Hinkkanen, 2007). These viruses belong to various virus classes and families.

Viruses from the family paramyxoviridae that have been tested as oncolytic agents include measles virus (eg. (Peng et al., 2001), Newcastle disease virus (eg. (Sinkovics and Horvath, 2000), Tupaia paramyxovirus (Springfeld et al., 2005), mumps virus (eg. (Myers et al., 2005), simian virus 5 (eg. (Parks et al., 2002) and sendai virus (eg. (Kinoh et al., 2004).

In (Stojdl et al., 2003) it is describe that in the range of 80% of all tested tumor cell lines, there is a defect in the interferon response following infection with Vesicular Stomatitis Virus (VSV). It may be assumed that a similar percentage of tumor cell lines will be susceptible to infection with NDV because both VSV and NDV are members of the order mononegavirales. It has also been shown that the mechanism of selective replication of NDV in tumor cells is based on a defect in the cellular interferon response against the virus (see for example WO 99/18799). What concerns recombinant paramyxoviruses, in EP-A-0702085 the genetically manipulated infectious replicating non-segmented negative-stranded RNA virus mutants, comprising an insertion and/or deletion in an open reading frame, a pseudogen region or an intergenic region of the virus genome is described.

Further, in WO99/66045 genetically modified NDV viruses obtained from full-length cDNA molecules of the virus genome are described.

In WO 00/62735 a method of tumor treatment comprising administering an interferon-sensitive, replication competent clonal RNA virus, such as NDV is described. Further descried is the use of avian paramyxovirus 2. However, no supporting data are available with regard that APMV2 has oncolytic activity.

In WO 01/20989 (PCT/US00/26116) a method for treating patients having tumor with recombinant oncolytic paramyxoviruses is described. The tumor is reduced by administering a replication competent paramyxoviridae virus. Various methods are described that can be used to engineer the virus genome in order to improve the oncolytic properties.

Further, in WO 03/005964 recombinant VSV comprising a nucleic acid encoding a cytokine is described.

In U.S. Pat. No. 6,699,479 NDV mutants are described which express the V-protein at a reduced level and comprising nucleotide substitutions in an editing locus.

In US 2004/0170607 the treatment of melanoma by administering a virus which is not a common human pathogen is described.

In WO2006/50984 recombinant NDV virus is described together with gene coding for anti tumor proteins. Especially a recombinant RNA-virus, preferably a paramyxovirus, preferably Newcastle disease virus (NDV) for treatment of diseases, especially for oncolytic tumor treatment is described. It is further described that recombinant viruses are produced that encode binding proteins (antibodies, ankyrin repeat molecules, peptides etc.), prodrug-converting enzymes and/or proteases and which lead to the selective expression of these molecules in virus-infected tumor cells. The activity of these binding proteins, prodrug-converting enzymes and/or proteases increases the anti-tumor effect of the virus. In WO2006/50984 the manufacture and the use of such modified viruses for treatment of cancer is described.

NDV can be genetically manipulated using the reverse genetics technology as described e.g. in EP-A-0702 085. For example, it is known to make recombinant NDV constructs comprising additional nucleic acids coding for secreted alkaline phosphatase (Zhao and Peeters, 2003), green fluorescent protein (Engel-Herbert et al., 2003), VP2 protein of infectious bursal disease virus (Huang et al., 2004), influenza virus hemagglutinin (Nakaya et al., 2001) and chloramphenicol acetyl transferase

(Huang et al., 2001) (Krishnamurthy, Huang, and Samal, 2000). None of these recombinant NDV has been constructed for use in the treatment of human disease. The recombinant NDVs were made to study either basic virology of NDV or to develop vaccine strains for poultry. As parental virus strains served lentogenic strains of NDV. These strains do not have significant oncolytic properties.

Even with respect to the known virus systems and processes, there is still a huge demand for those virus species which can selectively be used for the treatment of cancer, respectively tumors, and which show a superiority over the known virus specimens.

Moreover, there are no data available in the art which show that APMV3-9 can be used for the production of a medicament for the treatment of cancer, respectively tumors.

It has now surprisingly be found that selective virus specimens of the instant invention, derived from the family of paramyxovirus, derived from the sub-class of APMV3-9 can be used in tumor therapy with a surprising effective reactivity in contrast to the state of the art specimens.

Paramyxovirus of the instant invention have not been described in the art to have an antitumor effect.

Those virus selected from the family of paramyxovirus are, for example, Avian paramyxovirus 3 (APMV3), Avian paramyxovirus 4 (APMV4), Avian paramyxovirus 5 (APMV5), Avian paramyxovirus 6 (APMV6), Avian paramyxovirus 7 (APMV7), Avian paramyxovirus 8 (APMV8) and Avian paramyxovirus 9 (APMV9), as well as Mapueravirus and Fer-de-Lance virus.

The viruses APMV3-9 are described in (Alexander, 2003).

The invention further comprises a process for the production of a chimeric virus by molecular biologically combining genetic elements of different viruses.

Especially the invention comprises the use of paramyxovirus from the group selected from APMV3, APMV4, APMV5, APMV6, APMV7, APMV8, APMV9, Mapueravirus and Fer-de-Lance virus for the production of a medicament for the treatment of cancer, respectively tumors.

Treatment of cancer and tumors means inhibition of tumor growth, preferably the killing of the tumor cells or the blocking of proliferation in a time gap by infection. The described paramyxovirus replicates selectively in tumor cells.

Especially the use comprises the production of a medicament for the treatment of proliferative disorders, in particular hyperproliferative disorders. Preferably neoplasms can be treated with the described virus, preferably cancers from the group consisting of lung, colon, prostate, breast and brain cancer can be treated.

More preferably the invention concerns the use of the inventive virus for the production of a medicament for the treatment of a solid tumor and metastatic tumor.

More preferably a tumor with low proliferation rate can be treated.

Examples of tumors with low proliferation rate are prostate cancer, breast cancer, lung cancer, ovarian cancer, melanoma, cervical cancer, bladder cancer, glioblastoma and fibrosarcoma.

In a further selected aspect the invention concerns the use of the virus for the production of a medicament for the treatment of brain tumors and glioblastoma.

A further aspect of the instant invention is a closely related paramyxovirus that has more than 80% sequence identity on RNA level. That virus can be a newly discovered paramyxovirus.

The inventive virus may further be recombinant and may further be modified to express one or more additional genes that originates from APMV1-9, preferably it may be modified to express one or more additional gene(s) that originates from APMV1 (Newcastle disease virus).

The inventive virus may further be modified to comprise a gene encoding for a binding protein (see WO2006/050984).

The additional gene may encode for an enzyme, especially a prodrug converting enzyme, for an antibody or a fusion protein comprising at least one immunoglobulin domain with an antibody variable region (see WO2006/050984)

The modification results in a higher oncolytic potency as measured by the antitumor-effect when administered to tumor-bearing human or animal, for example a nude mice.

The inventive virus may further encode the gene(s) for the F and/or the HN protein of another paramyxovirus, whereby the F protein may have a multibasic cleavage site, and the virus may be modified in such a way that one gene is replaced by the homologous gene of a virus from the group APMV1-9.

The genetic modification may further result in an attenuation of the virus pathogenicity in birds.

The invention further comprises the use of the virus for the production of a medicament for the treatment of the above mentioned diseases together with pharmaceutically acceptable carrier and diluents. Such carrier and diluents are described in Remington's Pharmaceutical Science, 15^{th} ed. Mack Publishing Company, Easton Pa. (1980). The used virus titers may be in the range of 10⁹ to 10¹² pfu per dose, in a range of 10⁸ to 10¹¹ pfu, in a range of 10⁷ to 10¹⁰ pfu or in a range of 10⁶ to 10⁹ pfu dependent on the indication of treatment.

The pharmaceutical carrier and diluents may comprise an emulsion of the inventive virus, and may be administered by inhalation, intravenous infusion, subcutaneous injection, intraperitoneal injection or intratumoral injection.

Yet another aspect of the present invention is a method for the prevention or/and treatment of a proliferative disorder, in particular cancer, comprising administration to a subject in need thereof a pharmaceutically effective amount of the pharmaceutical composition of the present invention. A pharmaceutically effective amount is a titer of the virus of the present invention, in particular the virus of the present invention which cures or suppresses the disease.

For the therapeutic effect the acceptable dosis is different and depends for example from the construct, the patient, the ways of administration and the type of cancer.

The invention further comprises the use of the inventive virus in combination with a chemotherapeutic agent.

The inventive virus may be used with any anti-tumor agents, alkylating agents, antimetabolites, plant-derived anti-tumor agents, hormonal therapy agents, topoisomerase inhibitors, camptothecin derivatives, kinase inhibitors, targeted drugs, antibodies, interferons and/or biological response modifier and other anti-tumor-drugs. In this regard, the following is a non-limiting list of examples of secondary agents that may be used with the virus of the invention:

Alkylating agents include, but are not limited to, nitrogen mustard N-oxide, cyclophophamide, ifosfamide, thiotepa, ranimustine, nimustine, temozolomide, altretamine, apaziquone, brostallicin, bendamustine, carmustine, estramustine, fotemustine, glufosfamide, ifosfamide, mafosfamide, bendamustin and mitolactol;
platinum-coodinated alkylating compounds include but are not limited to, cisplatin, carboplatin, eptaplatin, lobaplatin, nedaplatin, oxaliplatin or satrplatin;
Antimetabolites include but are not limited to, methotrexate, 6-mercaptopurine riboside, mercaptopurine, 5-fluorouracil alone or in combination with leucovorin, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, vinorelbine,
Hormonal therapy agents, e.g., exemestane, Lupron, anastrozole, doxercalciferol, fadrozole, formestane, 11 Beta-Hydroxysteroid Dehydrogenase 1 inhibitors, 17-Alpha Hydroxylase/17,20 Lyase Inhibitors such as abiraterone acetate, 5-Alpha Reductase Inhibitors such as Bearfina (finasteride) and Epristeride, anti-estrogens such as tamoxifen citrate and fulvestrant, Trelstar, toremifene, raloxifene, lasofoxifene, letrozole, or anti-androgens such as bicalutamide, flutamide, mifepristone, nilutamide, Casodex, or anti-progesterones and combinations thereof;
Plant derived anti-tumor substances include for example those selected from mitotic inhibitors, for example epothilone such as sagopilone, Ixabepilone or epothilone B, vinblastine, vinflunine, docetaxel and paclitaxel;
Cytotoxic topoisomerase inhibiting agents include one or more agents selected from the group consisting of aclarubicin, amonafide, belotecan, camptothecin, 10-hydroxycamptothecin, 9-aminocamptothecin, diflomotecan, irinotecan (Camptosar), edotecarin, epimbicin (Ellence), etoposide, exatecan, gimatecan, lurtotecan, mitoxantrone, pirambicin, pixantrone, rubitecan, sobuzoxane, tafluposide, and topotecan, and combinations thereof;
Immunologicals include interferons and numerous other immune enhancing agents.
Interferons include interferon alpha, interferon alpha-2a, interferon, alpha-2b, interferon beta, interferon gamma-1a or interferon gamma-n1. Other agents include L19-IL2 and other L19 derivatives, filgrastim, lentinan, sizofilan, TheraCys, ubenimex, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, Provenge,
Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ProMune or ubenimex.
Pro-apoptotic agents are YM155, AMG 655, APO2L/TRAIL, CHR-2797.
Anti-angiogenic compounds include, acitretin, Aflibercept, angiostatin, aplidine, asentar, Axitinib, Recentin, Bevacizumab, brivanib alaninat, cilengtide, combretastatin, DAST, endostatin, fenretinide, halofuginone, pazopanib, Ranibizumab, rebimastat, removab, Revlimid, Sorafenib, Vatalanib, squalamine, Sunitinib, Telatinib, thalidomide, ukrain, Vitaxin,
Platinum-coordinated compounds include but are not limited to, cisplatin, carboplatin, nedaplatin, satraplatin or oxaliplatin;
Camptothecin derivatives include but are not limited to camptothecin, 10-hydroxycamptothecin, 9-aminocamptothecin, irinotecan, edotecarin, and topotecan;
Antibodies include Trastuzumab, Cetuximab Bevacizumab, or Rituximab, ticilimumab, Ipilimumab, lumiliximab, catumaxomab, atacicept; oregovomab, alemtuzumab;
VEGF inhibitors can also be combined with the inventive virus. Examples of VEGF inhibitors are Sorafenib, DAST, Bevacizumab, Sunitinib, Recentin, Axitinib, Aflibercept, Telatinib, brivanib alaninate, Vatalanib, pazopanib and Ranibizumab.
EGFR (HER1) inhibitors can also be combined with the inventive virus. Examples of EGFR inhibitors are Cetuximab, Panitumumab, Vectibix, Gefitinib, Erlotinib, Zactima.
HER2 inhibitors can also be combined with the inventive virus. Examples of HER2 inhibitors are Lapatinib, Tratuzumab, Pertuzumab.
mTOR inhibitors can also be combined with the inventive virus. Examples of mTOR inhibitors are Temsirolimus, sirolimus/Rapamycin, everolimus.
cMet inhibitors can also be combined with the inventive virus.
PI3K− and AKT inhibitors can also be combined with the inventive virus.
CDK inhibitors can also be combined with the inventive virus. Examples of CDK inhibitors are roscovitine and flavopiridol.
Spindle assembly checkpoints inhibitors and targeted anti-mitotic can be combined with the inventive virus. Example for targeted anti-mitotic drug are the PLK inhibitors, the Aurora inhibitors such as Hesperadin, Checkpoint Kinase inhibitors and the KSP inhibitors.
HDAC inhibitors can be combined with the inventive virus. Example for HDAC inhibitors are panobinostat, vorinostat, MS275, belinostat and LBH589.
HSP90 inhibitors and HSP70 inhibitors can be combined with the inventive virus.
Proteasome inhibitors can be combined with the inventive virus. Examples for proteasome inhibitors are bortezomib and carfilzomib.
Serine/threonine kinase inhibitors can be combined with the inventive virus. Serine kinase inhibitors include MEK inhibitors and Raf inhibitors such as Sorafenib.
The inventive virus may be used with Farnesyl transferase inhibitors, e.g. tipifarnib.
The inventive virus may be used with tyrosine kinase inhibitors including Dasatinib, Nilotibib, DAST, Bosutinib, Sorafenib, Bevacizumab, Sunitinib, AZD2171, Axitinib, Aflibercept, Telatinib, imatinib mesylate, brivanib alaninate, pazopanib, Ranibizumab, Vatalanib, Cetuximab, Panitumumab, Vectibix, Gefitinib, Erlotinib, Lapatinib, Tratuzumab, Pertuzumab and c-Kit inhibitors.
Vitamin D receptor agonists can be combined with the inventive virus.
Bcl-2 protein inhibitors can be combined with the inventive virus. Example for Bcl-2 protein inhibitors are obatoclax, oblimersen sodium and gossypol.
Cluster of Differentiation 20 receptor antagonists can be combined with the inventive virus. An example for a Cluster of Differentiation 20 receptor antagonist is rituximab.
Ribonucleotide Reductase Inhibitors can be combined with the inventive virus. An example for a Ribonucleotide Reductase Inhibitor is Gemcitabine.
Topoisomerase I and II Inhibitors can be combined with the inventive virus. Example for a Topoisomerase I and II Inhibitor is Camptosar (Irinotecan) and doxorubicin.
Tumor Necrosis Apoptosis Inducing Ligand Receptor 1 Agonists can be combined with the inventive virus. Example for a Tumor Necrosis Apoptosis Inducing Ligand Receptor 1 Agonist is mapatumumab.
5-Hydroxytryptamine Receptor Antagonists can be combined with the inventive virus. Example for 5-Hydroxytryptamine Receptor Antagonists are rEV598, Xaliprode, Palonosetron hydrochloride, granisetron, Zindol, palonosetron hydrochlorid or AB-1001.
Integrin Inhibitors can be combined with the inventive virus. Example for Integrin Inhibitors are Alpha5Beta1 integrin inhibitors such as E7820, JSM 6425, volociximab or Endostatin.
Androgen receptor antagonists can be combined with the inventive virus. Examples for Androgen receptor antagonists are nandrolone decanoate, fluoxymesterone, fluoxymesterone, Android, Prost-aid, Andromustine, Bicalutamide, Flutamide, Apo-Cyproterone, Apo-Flutamide, chlormadinone acetate, bicalutamide, Androcur, Tabi, cyproterone acetate, Cyproterone Tablets, nilutamide.
Aromatase Inhibitors can be combined with the inventive virus. Examples for Aromatase Inhibitors are anastrozole, letrozole, testolactone, exemestane, Aminoglutethimide and formestane.
Matrix metalloproteinase inhibitors can be combined with the inventive virus.
Other anticancer agents include alitretinoin, ampligen, atrasentan bexarotene, bortezomib, bosentan, calcitriol, exisulind, finasteride, fotemustine, ibandronic acid, miltefosine, mitoxantrone, I-asparaginase, procarbazine, dacarbazine, hydroxycarbamide, hydroxycarbamide, pegaspargase, pentostatin, tazarotne, velcade, gallium nitrate, Canfosfamide darinaparsin or tretinoin;

### EXAMPLES

### Tumor-Selective Replication

When incubated with human tumor cells such as HT29 colon carcinoma cells or HT1080 fibrosarcoma cells, the virus is able to replicate and produce progeny virus. The titer achieved in tumor cells is at least 100 fold higher than the titer achieved in human primary normal cells.

When the cell-killing effect of the virus is examined the necessary MOI to kill primary normal cells is at least 100, more preferably 1000 fold higher than the MOI that is sufficient to kill human tumor cells.

### Production of the Virus

The virus is grown either in embryonated bird eggs (preferably chicken eggs) or in human tumor cell lines or derivatives of human tumor cell lines.

The virus is harvested from the allantoic fluid of the eggs or from the supernatant of the cells or from the cell pellet (cell-associated virus).

The virus is concentrated and purified using a discontinuous sucrose gradient ultracentrifugation. Alternatively the virus is concentrated and purified using tangential flow filtration.

### Anti-Tumor Effect

The anti-tumor effect of the virus is shown in murine tumor models. When administered intratumorally or intravenously, the virus results in the regression of established human xenograft-tumors in nude mice. The effective dose of virus is in the range of 10⁵ to 10⁹ pfu/injection. In some tumor models repeated administration in intervals of 2-14 days (preferably 7 days) is necessary to be most effective.

### Genetic Engineering of the Virus

The reverse genetic system is published for many paramyxoviruses. It is adapted analogously to the paramyxovirus of this invention.

The viral genome is sequenced. Based on the sequence, primers for cloning are designed that span unique restriction enzyme recognition sites within the viral genome. By RT-PCR several fragments of the viral genome are cloned as DNA in a plasmid vector like pX8δT.

With the genomic plasmid, mutations, exchange of genes and other types of genetic engineering are carried out on the DNA-level.

By transfection of the genomic plasmid-DNA into cells that express the T7-polymerase together with helper plasmids, recombinant virus can be rescued. An alternative method for the rescue of virus may be used. For example expression of the T7-polymerase may be accomplished by cotransfecting the cells with an expression plasmid encoding for T7-polymerase.

Additional transgenes are expressed in the virus as it is described for the related virus NDV (Puhler et al., 2008).

The additional transgenes are preferably inserted in the intergenic region between M-F or F-HN.

The F and HN transgenes may be derived from any APMV, preferably from a virus that has itself strong oncolytic potency. The F and H/HN transgenes may be derived from another paramyxovirus other than APMV.

It may be sufficient to express only the heterologous F or the HN protein individually in order to increase the oncolytic potency of the virus.

### Chimeric Viruses

In order to combine positive features of two different viruses or in order to get rid of negative features it is possible to substitute fragments of the genome with homologous fragments of another virus. That virus may be any related paramyxovirus and is not limited to APMV.

The endogenous genes for the F and HN proteins of APMV can be replaced by heterologous genes from related paramyxoviruses to alter the specificity and tumor-selectivity of the virus.

To allow proper assembly of virions all three proteins M, F and HN can be replaced by the homologous gene segment of a related virus.

In order to decrease the pathogenicity for birds, the gene for the P/V protein is either mutated or replaced by the gene from a related virus. The same procedure can also increase the specificity of the virus for killing of human tumor cells compared to normal cells.

The endogenous L, P and NP proteins can be exchanged. The exchange of these three proteins will have an effect on the replication properties of the virus.

### Transgenes

In order to decrease the pathogenicity of the virus, the gene for avian interferon is inserted into the viral genome.

In order to increase the tumor-penetration of the virus, transgene(s) are inserted that encode for extracellular-matrix-resolving enzymes like relaxin, collagenase, MMP etc.

In order to increase the therapeutic potency, transgenes are inserted that encode for proteins that have an ant-tumor activity like toxins, prodrug-converting enzymes, proteases, antibodies etc. Examples are TRAIL and mutants thereof, MDA-7, IL2, TNF-a, IGF-BP-7.

For imaging purposes and as biomarkers, transgene(s) are inserted that encode for reporter-genes like GFP, luciferase, NIS or marker peptides like PSA, insulin C-peptide, common virus-antigens (peptides) etc.

### REFERENCES

Alexander, D. J. (2003). Newcastle Disease, Other Avian Paramyxoviruses, and Pneumovirus Infections. ISBN 081380423X ed. In “Diseases of Poultry” (Y. M. Saif, Ed.), pp. 63-99. Blackwell Publishing.
Chiocca, E. A. (2002). Oncolytic viruses. Nat Rev Cancer 2(12), 938-50.
Engel-Herbert, I., Werner, O., Teifke, J. P., Mebatsion, T., Mettenleiter, T. C., and Romer-Oberdorfer, A. (2003). Characterization of a recombinant Newcastle disease virus expressing the green fluorescent protein. J Virol Methods 108(1), 19-28.
Huang, Z., Elankumaran, S., Yunus, A. S., and Samal, S. K. (2004). A recombinant Newcastle disease virus (NDV) expressing VP2 protein of infectious bursal disease virus (IBDV) protects against NDV and IBDV. J Virol 78(18), 10054-63.
Huang, Z., Krishnamurthy, S., Panda, A., and Samal, S. K. (2001). High-level expression of a foreign gene from the most 3′-proximal locus of a recombinant Newcastle disease virus. J Gen Virol 82(Pt 7), 1729-36.
Kinoh, H., Inoue, M., Washizawa, K., Yamamoto, T., Fujikawa, S., Tokusumi, Y., Iida, A., Nagai, Y., and Hasegawa, M. (2004). Generation of a recombinant Sendai virus that is selectively activated and lyses human tumor cells expressing matrix metalloproteinases. Gene Ther 11(14), 1137-45.
Krishnamurthy, S., Huang, Z., and Samal, S. K. (2000). Recovery of a virulent strain of newcastle disease virus from cloned cDNA: expression of a foreign gene results in growth retardation and attenuation. Virology 278(1), 168-82.
Myers, R., Greiner, S., Harvey, M., Soeffker, D., Frenzke, M., Abraham, K., Shaw, A., Rozenblatt, S., Federspiel, M. J., Russell, S. J., and Peng, K. W. (2005). Oncolytic activities of approved mumps and measles vaccines for therapy of ovarian cancer. Cancer Gene Ther 12(7), 593-9.
Nakaya, T., Cros, J., Park, M. S., Nakaya, Y., Zheng, H., Sagrera, A., Villar, E., Garcia-Sastre, A., and Palese, P. (2001). Recombinant Newcastle disease virus as a vaccine vector. J Virol 75(23), 11868-73.
Parks, G. D., Young, V. A., Koumenis, C., Wansley, E. K., Layer, J. L., and Cooke, K. M. (2002). Controlled cell killing by a recombinant nonsegmented negative-strand RNA virus. Virology 293(1), 192-203.
Peng, K. W., Ahmann, G. J., Pham, L., Greipp, P. R., Cattaneo, R., and Russell, S. J. (2001). Systemic therapy of myeloma xenografts by an attenuated measles virus. Blood 98(7), 2002-7.
Puhler, F., Willuda, J., Puhlmann, J., Mumberg, D., Romer-Oberdorfer, A., and Beier, R. (2008). Generation of a recombinant oncolytic Newcastle disease virus and expression of a full IgG antibody from two transgenes. Gene Ther.
Sinkovics, J. G., and Horvath, J. C. (2000). Newcastle disease virus (NDV): brief history of its oncolytic strains. J Clin Virol 16(1), 1-15.
Springfeld, C., von Messling, V., Tidona, C. A., Darai, G., and Cattaneo, R. (2005). Envelope targeting: hemagglutinin attachment specificity rather than fusion protein cleavage-activation restricts Tupaia paramyxovirus tropism. J Virol 79(16), 10155-63.
Stojdl, D. F., Lichty, B. D., tenOever, B. R., Paterson, J. M., Power, A. T., Knowles, S., Marius, R., Reynard, J., Poliquin, L., Atkins, H., Brown, E. G., Durbin, R. K., Durbin, J. E., Hiscott, J., and Bell, J. C. (2003). VSV strains with defects in their ability to shutdown innate immunity are potent systemic anti-cancer agents. Cancer Cell 4(4), 263-75.
Vaha-Koskela, M. J., Heikkila, J. E., and Hinkkanen, A. E. (2007). Oncolytic viruses in cancer therapy. Cancer Lett.
Zhao, H., and Peeters, B. P. (2003). Recombinant Newcastle disease virus as a viral vector: effect of genomic location of foreign gene on gene expression and virus replication. J Gen Virol 84(Pt 4), 781-8.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding EP application No. 08075121.7, filed Feb. 14, 2008, and U.S. Provisional Application Ser. No. 61/029,650, filed Feb. 19, 2008, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for tumor treatment comprising administering paramyxovirus from the group APMV3, APMV4, APMV5, APMV6, APMV7, APMV8, APMV9, Mapueravirus or Fer-de-Lance virus.

2. Method according to claim 1, wherein the virus is a closely related paramyxovirus that has more than 80% sequence identity on RNA level.

3. Method according to claim 1, wherein the virus is recombinant.

4. Method according to claim 1, wherein the virus is modified to express one additional gene that originates from APMV1-9.

5. Method according to claim 1, wherein the virus is modified to express two or more additional genes that originate from APMV1-9

6. Method according to claim 1, wherein the genes encode the F and/or the HN protein of another paramyxovirus.

7. Method according to claim 6, wherein the F protein has a multibasic cleavage site.

8. Method according to claim 3, wherein the virus is modified in such a way that one gene is replaced by the homologous gene of a virus from the group APMV1-9

9. Method according to claim 3, wherein the genetic modification results in an attenuation of pathogenicity in birds.

10. Method according to claim 3, wherein the genetic modification results in a higher selectivity of the virus to infect tumor cells compared to non-transformed normal cells.

11. Method according to claim 3, wherein the genetic modification results in a higher oncolytic potency as measured by the antitumor-effect when administered to tumor-bearing nude mice.

12. Method according to claim 1, wherein the virus is administered intratumorally.

13. Method according to claim 1, wherein the virus is administered intraperitoneally.

14. Method according to claim 1, wherein the virus is administered by inhalation.

15. Method according to claim 1, wherein the virus is administered intravenously.

16. Method according to claim 1, wherein the virus is purified by gradient ultracentrifugation.

17. Method according to claim 1, wherein the virus is purified by tangential flow filtration.

18. Method according to claim 1, wherein the tumor is selected out of the group consisting of colon carcinoma, breast carcinoma, lung carcinoma, prostate carcinoma, ovarian carcinoma, melanoma, cervical carcinoma, bladder carcinoma, glioblastoma and fibrosarcoma.

19. Method according to claim 1, wherein the pharmaceutical composition further comprises a chemotherapeutic agent.

20. Method according to claim 1, wherein the said pharmaceutical composition further comprises a recombinant therapeutic antibody.

21. Method according to claim 1, wherein the pharmaceutical composition further comprises a recombinant therapeutic protein.

22. Method according to claim 1, wherein the virus is modified to express at least one additional gene encoding for a binding protein.

23. Method according to claim 1, wherein the virus is modified to express at least one additional gene encoding for an enzyme.

24. Method according to claim 1, wherein the virus is modified to express at least one additional gene encoding for a prodrug converting enzyme.

25. Method according to claim 1, wherein the virus is modified to express at least one additional gene encoding for an antibody.

26. Method according to claim 3, wherein the virus is modified to express at least one additional gene encoding for a fusion protein comprising at least one immunoglobulin domain with an antibody variable region.

27. Method according to claim 1, wherein the tumor is metastatic.

28. Method according to claim 8, wherein two to five genes are replaced, resulting in a chimeric virus that has only one to four remaining genes of the originating virus.
